(19) United States
(12) Patent Application Publication
(10) Pub. No.: US 2019/0008571 A1
(43) Pub. Date: Jan. 10, 2019

(54) BONE SCREW HAVING A DRILL BIT PORTION

(71) Applicant: MIRUS LLC, Atlanta, GA (US)

(72) Inventor: Noah Roth, Atlanta, GA (US)

(21) Appl. No.: 16/080,371

(22) PCT Filed: Feb. 28, 2017
(86) PCT No.: PCT/US17/19921

§ 371 Date: Aug. 28, 2018

Related U.S. Application Data
(60) Provisional application No. 62/301,158, filed on Feb. 29, 2016.

(51) Int. Cl. A61B 17/86; A61B 17/16

(52) U.S. Cl. CPC A61B 17/8635; A61B 17/863; A61B 17/1615

(57) ABSTRACT

An example bone screw is described herein. The bone screw can include a drill bit portion configured to create a pilot hole in a bone of a subject, and a threaded portion arranged proximally with respect to the drill bit portion. The threaded portion can be configured for securing the bone screw in the bone of the subject. Additionally, an outer diameter of the drill bit portion is smaller than an outer diameter of the threaded portion.

### CROSS-REFERENCE TO RELATED APPLICATIONS

This application claims the benefit of U.S. Provisional Patent Application No. 62/301,158, filed on Feb. 29, 2016, entitled “BONE SCREW HAVING A DRILL BIT PORTION,” the disclosure of which is expressly incorporated herein by reference in its entirety.

### BACKGROUND

Bone screws are commonly used in spinal surgery. For example, a pedicle screw is a bone screw inserted into a pedicle of the spine. Pedicle screws are commonly used during spinal fusion surgery. For example, a plurality of pedicle screws, which act as firm anchor points, are inserted into a subject's spine, and a rod is used to connect the pedicle screws. The pedicle screws and rod together prevent motion of portions of the subject's spine.

### SUMMARY

An example bone screw is described herein. The bone screw can include a drill bit portion configured to create a pilot hole in a bone of a subject, and a threaded portion arranged proximally with respect to the drill bit portion. The threaded portion can be configured for securing the bone screw in the bone of the subject. Additionally, an outer diameter of the drill bit portion is smaller than an outer diameter of the threaded portion.

The outer diameter of the drill bit portion can be about 25% smaller than the outer diameter of the threaded portion. Optionally, the outer diameter of the drill bit portion can be from about 25% to about 30% smaller than the outer diameter of the threaded portion.

Alternatively or additionally, the drill bit portion can include a first elongate cylinder, where the first elongate cylinder defines the outer diameter of the drill bit portion. Alternatively or additionally, the threaded portion can include a second elongate cylinder, where the second elongate cylinder defines the outer diameter of the threaded portion.

Alternatively or additionally, the bone screw can include an intermediate tapered portion arranged between the drill bit portion and the threaded portion.

Alternatively or additionally, the drill bit portion can include a spiral flute extending axially from about a distal end of the drill bit portion. The spiral flute can include a cutting edge configured to remove the bone of the subject. Alternatively or additionally, the drill bit portion can include a distal tip configured to create a starter hole.

Alternatively or additionally, the threaded portion can include a spiral thread extending axially from about a distal end of the threaded portion.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a view of an example bone screw.

FIG. 2 is an end view of the bone screw of FIG. 1 at the distal tip.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for a bone screw, it will become evident to those skilled in the art that the implementations are not limited thereto.

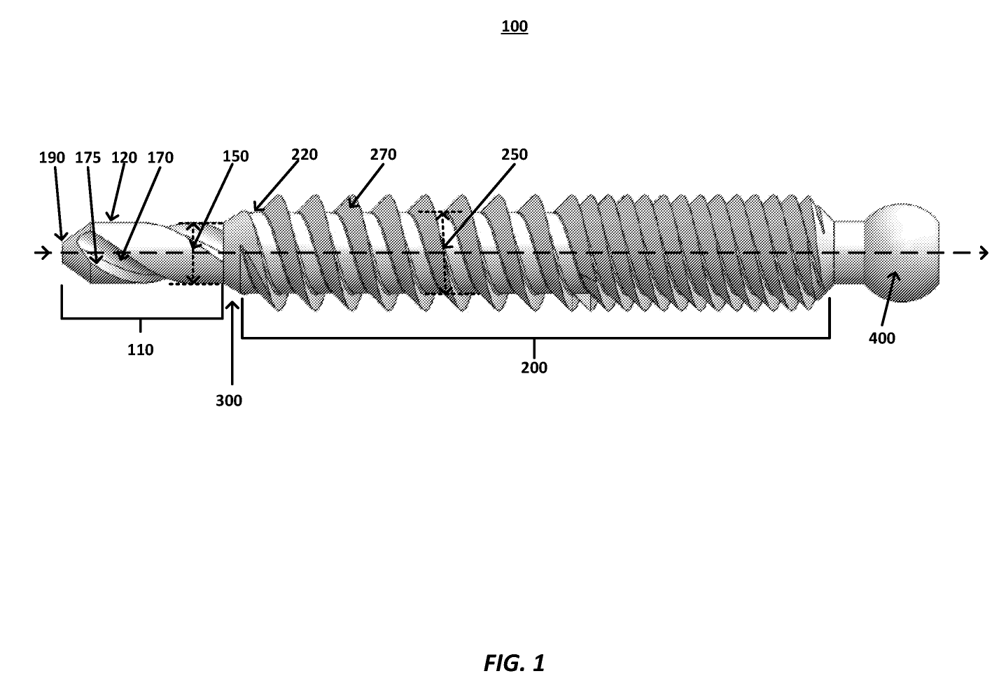

As used herein, distal refers to the direction towards which a bone screw advances as the bone screw is inserted into bone. Proximal refers to the direction opposite of the distal direction. Additionally, axial refers to a direction along an axis of the bone screw, which is shown by the dotted line in FIG. 1. Radial refers to a direction along a radius of a cross section of the bone screw.

Referring now to FIG. 1, an example bone screw 100 is shown. The bone screw 100 can include a drill bit portion 110 configured to create a pilot hole in a bone of a subject, and a threaded portion 200 arranged proximally with respect to the drill bit portion 110. The threaded portion 200 can be configured for securing the bone screw 100 in the bone of the subject. Additionally, an outer diameter of the drill bit portion 110 is smaller than an outer diameter of the threaded portion 200. In FIG. 1, the outer diameter of the drill bit portion 110 is shown by reference number 150, and the outer diameter of the threaded portion 200 is shown by reference number 250. The outer diameter of the drill bit portion 110 can be about 25% smaller than the outer diameter of the threaded portion 200. Optionally, the outer diameter of the drill bit portion 110 can be from about 25% to about 30% smaller than the outer diameter of the threaded portion 200.

This disclosure contemplates that the bone screw 100 can be inserted with less steps than required in conventional procedures. In conventional procedures, multiple steps may include creating a starter hole with a small drill bit, creating a pilot hole with a larger drill bit, tapping the pilot hole, and/or inserting the bone screw. As shown in FIG. 1, the bone screw 100 includes the drill bit portion 110 configured to create the pilot hole, and the threaded portion 200 configured for securing the bone screw 100 in the bone of the subject. Accordingly, a surgeon or other medical professional does not need to switch instrumentation while inserting the bone screw 100. Additionally, the respective outer diameters of the drill bit portion 110 and the threaded portion 200 are different. In particular, the outer diameter of the drill bit portion 110 is smaller (i.e., less) than the outer diameter of the threaded portion 200. As a result, the threaded portion 200 is driven into the pilot hole created by the drill bit portion 110 (i.e., the portion having a smaller diameter), where the pilot hole has a diameter less than the threaded portion 200. The bone surrounding the pilot hole compresses the threaded portion 200 of the bone screw 100. This tends to more securely fix the bone screw 100 in the bone of the subject as compared to a case where a bone screw has drill bit and threaded portions with equal diameter.

Alternatively or additionally, the drill bit portion 110 can include a first elongate cylinder 120, where the first elongate cylinder 120 defines the outer diameter of the drill bit portion 110. As described below, a spiral flute 170 forms a cavity or channel within the first elongate cylinder 120. Alternatively or additionally, the threaded portion 200 can include a second elongate cylinder 220, where the second elongate cylinder 220 defines the outer diameter of the threaded portion 200. As described below, a spiral thread 270 extends radially outward from the second elongate cylinder 220.

Alternatively or additionally, the bone screw 100 can include an intermediate tapered portion 300 arranged between the drill bit portion 110 and the threaded portion 200. A diameter of the intermediate tapered portion 300 tapers from a maximum diameter about equal to the outer diameter of the threaded portion 200 (i.e., shown by reference number 250) to a minimum diameter about equal to the outer diameter of the drill bit portion 110 (i.e., shown by reference number 150). Alternatively or additionally, the bone screw 100 can include a head 400 arranged proximally with respect to the threaded portion 200.

Alternatively or additionally, the drill bit portion 110 can include a spiral flute 170. The spiral flute 170 forms a cavity or channel in the first elongate cylinder 120 of the drill bit portion 110. The spiral flute 170 runs in a spiral configuration up the first elongate cylinder 120 as shown in FIG. 1. The spiral flute 170 extends axially from about a distal end of the drill bit portion 110 to about a proximal end of the drill bit portion 110. Optionally, as shown in FIG. 1, the spiral flute 170 extends from about a distal end of the drill bit portion 110 to about a distal end of the intermediate tapered portion 300. The spiral flute can include a cutting edge 175 configured to remove the bone of the subject. Alternatively or additionally, the drill bit portion 110 can include a distal tip 190 configured to create a starter hole. The distal tip 190 can act as a sharper point at the terminal end of the bone screw 100 (i.e., the distal end of the bone screw 100). Alternatively or additionally, the distal tip 190 can have an angle that creates a blunt tip (e.g., as shown in FIG. 1) for contacting the bone of the subject. The blunt tip facilitates correct positioning of the drill bit portion 110 prior to drilling/insertion, which allows the surgeon or other medical professional to obtain the correct or desired trajectory into the bone.

Alternatively or additionally, the threaded portion 200 can include a spiral thread 270. The spiral thread 270 secures the bone screw 100 into the bone of the subject. The spiral thread 270 extends radially outward from the second elongate cylinder 220 of the threaded portion 200. The spiral thread 270 secures the bone screw 100 into the bone of the subject. The spiral thread 270 runs in a spiral configuration up the second elongate cylinder 220 as shown in FIG. 1. The spiral thread 270 extends axially from about a distal end of the threaded portion 200 to about a proximal end of the threaded portion 200. Optionally, as shown in FIG. 1, the spiral thread 270 extends from about a proximal end of the intermediate tapered portion 300 to about a proximal end of the threaded portion 200. Optionally, as shown in FIG. 1, characteristics of the spiral thread 270 (e.g., pitch, lead, angle, etc.) can vary within the threaded portion 200. For example, in FIG. 1, the pitch of a distal portion of the threaded portion 200 is greater than the pitch of a proximal portion of the threaded portion 200. It should be understood that this variation in characteristic of the spiral thread 270 is provided only as one example and is not intended to be limiting. In other words, in some implementations, characteristics of the spiral thread 270 are not variable within the threaded portion 200, while in other implementations, any one or more characteristics of the spiral thread 270 can vary within the threaded portion 200.

Optionally, the bone screw 100 can include a self-tapping feature and/or the spiral thread 270 can be self-tapping. Accordingly, a thread can be cut into the pilot hole created by the bone of the subject, and the spiral thread 270 can engage with the thread cut into the pilot hole created by the bone of the subject.

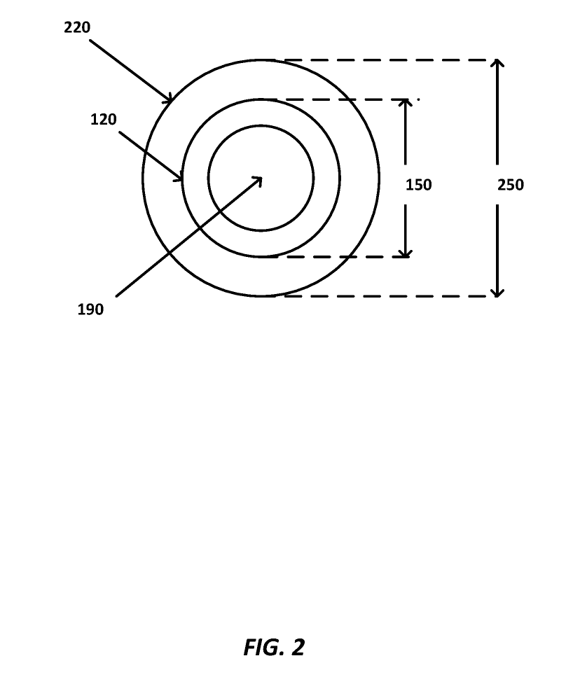

Referring now to FIG. 2, an end view of the bone screw of FIG. 1 at the distal tip 190 is shown. The first elongate cylinder 120 and the second elongate cylinder 220 are shown in FIG. 2. Additionally, the outer diameter of the drill bit portion is shown by reference number 150, and the outer diameter of the threaded portion is shown by reference number 250. As described above, the outer diameter of the drill bit portion can be about 25% smaller than the outer diameter of the threaded portion. Optionally, the outer diameter of the drill bit portion can be from about 25% to about 30% smaller than the outer diameter of the threaded portion.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A bone screw, comprising:
a drill bit portion configured to create a pilot hole in a bone of a subject; and
a threaded portion arranged proximally with respect to the drill bit portion, the threaded portion being configured for securing the bone screw in the bone of the subject, wherein an outer diameter of the drill bit portion is smaller than an outer diameter of the threaded portion.

2. The bone screw of claim 1, wherein the drill bit portion includes a first elongate cylinder, the first elongate cylinder defining the outer diameter of the drill bit portion.

3. The bone screw of claim 1, wherein the threaded portion includes a second elongate cylinder, the second elongate cylinder defining the outer diameter of the threaded portion.

4. The bone screw of claim 1, wherein the outer diameter of the drill bit portion is about 25% smaller than the outer diameter of the threaded portion.

5. The bone screw of claim 4, wherein the outer diameter of the drill bit portion is from about 25% to about 30% smaller than the outer diameter of the threaded portion.

6. The bone screw of claim 1, further comprising an intermediate tapered portion arranged between the drill bit portion and the threaded portion.

7. The bone screw of claim 1, wherein the drill bit portion comprises a spiral flute extending axially from about a distal end of the drill bit portion.

8. The bone screw of claim 7, wherein the spiral flute includes a cutting edge configured to remove the bone of the subject.

9. The bone screw of claim 1, wherein the drill bit portion comprises a distal tip configured to create a starter hole.

10. The bone screw of claim 9, wherein the distal tip comprises a blunt tip.

11. The bone screw of claim 1, wherein the threaded portion comprises a spiral thread extending axially from about a distal end of the threaded portion.
